(19) United States
(12) Patent Application Publication
(10) Pub. No.: US 2025/0235673 A1
(43) Pub. Date: Jul. 24, 2025

(54) CATHETER DEVICES WITH VALVES AND RELATED METHODS

(71) Applicant: B. Braun Melsungen AG, Melsungen (DE)

(72) Inventor: Boon Ping Neoh, Penang (MY)

(73) Assignee: B. Braun Melsungen AG, Melsungen (DE)

(21) Appl. No.: 18/880,933

(22) PCT Filed: Jul. 6, 2023
(86) PCT No.: PCT/EP2023/068670

§ 371 Date: Jan. 3, 2025

Related U.S. Application Data
(60) Provisional application No. 63/367,757, filed on Jul. 6, 2022.

(51) Int. Cl. A61M 25/06; A61M 25/00; A61M 39/22

(52) U.S. Cl. CPC A61M 25/0606; A61M 25/0097; A61M 25/0625; A61M 39/22; A61M 2039/226; A61M 2207/00

(57) ABSTRACT

Needle assemblies and related methods in which a valve opener is pushed into a valve to open one or more slits parting a plurality of flaps on the valve to open the valve, creating an internal reaction force within the plurality of flaps. The plurality of flaps of the valve utilize the internal reaction force to retract back to a closed position following removal of the valve opener from the valve, thereby sealing the valve until reopened if reused. The needle assemblies each include a needle hub with a needle, a catheter tube with a catheter hub, the valve, and the valve opener positioned in an interior cavity of the catheter hub.

### FIELD OF ART

The disclosed invention generally relates to needle devices, arterial catheters, and intravenous (IV) infusion devices, including IV catheters. Particularly, IV catheter assemblies having a valve and a valve actuator for opening the valve are disclosed.

### BACKGROUND

IV catheters are commonly used for a variety of infusion therapies, including infusing fluids into a patient, withdrawing blood from a patient, or monitoring various parameters of the patient's vascular system. Catheters are typically connected to a catheter adapter that accommodates the attachment of IV tubing to the catheter. Blood control catheters include an internal blood control valve that is opened by the insertion of a male Luer or other object into a proximal end of the catheter adapter. Non-limiting examples of blood control valves are disclosed in United States Patent Application Publication No. 2011/0046570, filed Aug. 20, 2009, titled “Systems and Methods for Providing a Flushable Catheter Assembly.” Following placement of the catheter into the vasculature of a patient, an IV fluid source can be connected to the catheter adapter or catheter hub, opening the blood control valve. Thus, fluid from the IV source can begin flow into a patient through the catheter.

As is well known in the art, typical blood pressure is 10 to 20 centimeters of water. Infusion bags are usually placed about 100 cm above the patient's heart to direct flow into the patient. At roughly that height, the pressure exerted by the fluid from the infusion bag is much greater than the blood pressure of the patient and therefore can flow into the patient.

Some catheter adapters permit verification of proper placement of the catheter in the blood vessel before fluid infusion begins, such as by providing a flashback chamber of the catheter assembly where a “flashback” of blood can be observed. To confirm flashback in catheter assemblies that do not include a blood control valve, a clinician must manually occlude the vein to prevent undesirable exposure to blood. In contrast, blood control valves can eliminate the need for such manual occlusion, while also reducing the likelihood of blood exposure during catheter placement.

### SUMMARY

An aspect of the present disclosure includes the provision for a multiple use blood control system having a reversible valve seal.

Another aspect of the present disclosure includes the provision for an intravenous catheter having a low-profile catheter hub.

A still further aspect of the present disclosure is the provision for improving the state of the art of blood control or closed system infusion devices including intravenous catheters.

As described, a needle assembly of the present disclosure may include a number of different components. The needle assembly may comprise a needle hub with a hub body and a needle with a needle tip extending from a distal end of the hub body; a catheter tube attached to a catheter hub and having the needle extending through the catheter tube in a ready to use position; a valve positioned in an interior cavity of the catheter hub, the valve comprising a valve body having an outer perimeter, an interior surface defining an interior cavity, a distal opening, a wall structure at a proximal end of the valve body, at least one slit and a plurality of flaps at the wall structure, and at least one interior bump on each of the plurality of flaps, each interior bump extending axially distally from a distally facing wall surface of the wall structure in interior cavity of the valve; a valve opener comprising a nose section with a push end sized and shaped to open the at least one slit to open the valve when displaced distally by a male Luer tip; and a needle guard positioned in the interior cavity of the catheter hub and placed over the needle.

The needle guard may comprise a wall positioned to a side of the needle and movable distal of the needle tip to block the needle tip from inadvertent needlesticks in a protective position.

In some examples, the valve may be practiced within the interior bumps only, without also the exterior bumps. In other examples, the valve may further comprise a plurality of exterior bumps extending in a proximal direction from a proximally facing surface of the wall structure.

Each of the plurality of flaps may have at least one exterior bump located thereon.

The valve may comprise a slanted wall surface located in the interior cavity for contacting by the interior bumps.

The interior cavity may have a first generally cylindrical wall section located adjacent the slanted wall surface. The interior cavity may have a second generally cylindrical wall section located adjacent to the slanted wall surface and remote from the first generally cylindrical wall section.

Either the interior bumps, the exterior bumps, or both the interior and exterior bumps on the wall structure at the proximal end of the valve, such as on the flaps of the wall structure, can extend the thickness of the wall structure. The thickness can be measured from the exterior most surface at the proximal side to the exterior most surface at the distal side of the wall structure, where the flaps are provided. When extending the thickness of the wall structure, such as the interior bumps, the flaps can be deflected by a valve opener to contact an interior surface of the valve with a male medical implement without having to deflect the flaps the same amount compared to when no bumps are incorporated.

The first generally cylindrical wall section may have a first inside diameter. The second generally cylindrical wall section may have a second inside diameter. The first inside diameter may be larger than the second inside diameter.

The valve may have a distal opening at a distal end of the valve body that opens into the second generally cylindrical wall section.

The at least one slit may comprise three slits. The plurality of flaps may comprise three flaps.

The valve opener may comprise two actuating elements having the needle guard located therebetween and a stabilizer connecting the two actuating elements.

A still further aspect of the present disclosure is a catheter assembly. The catheter assembly may comprise a needle hub with a needle having a needle tip; a catheter tube attached to a catheter hub having a body comprising an interior surface defining an interior cavity, the needle extending through the catheter tube; a valve positioned in the interior cavity of the catheter hub, the valve comprising a valve body having an exterior surface and an interior surface defining an interior cavity, a distal opening at a distal end of the valve body and a wall structure at a proximal end of the valve body, the wall structure comprising a plurality of slits defining a plurality of flaps; a plurality of interior bumps located on the flaps at a location inside the interior cavity of the valve and a plurality of exterior bumps located on the flaps at a location outside of the interior cavity of the valve, a valve opener located inside the interior cavity of the catheter hub and proximal of the valve, the valve opener comprising a nose section having an activation end, at least one plunger element having a proximal end surface for pushing by a male medical implement; a needle guard having a proximal wall and a blocking wall, wherein the blocking wall is positioned to a side of the needle and movable distal of the needle tip to block the needle tip from inadvertent needlesticks in a protective position.

At least one interior bump may be aligned with at least one exterior bump so that a thickness measured at the aligned at least one interior bump and at least one exterior bump is thicker than a thickness of the wall structure.

Each of the plurality of flaps may have at least one interior bump aligned with at least one exterior bump.

The valve may comprise a slanted wall surface located in the interior cavity for contacting by the interior bumps.

The interior cavity of the valve may have a first generally cylindrical wall section located adjacent the slanted wall surface and a second generally wall section located adjacent the slanted wall surface and remote from the first generally cylindrical wall section.

The valve may have a distal opening that opens into the second generally cylindrical wall section.

The valve opener may further comprise a stabilizing ring defining a continuous perimeter section.

A still further aspect of the present disclosure is a method of assembling a needle assembly. The method may comprise providing a catheter hub with a catheter tube having an open distal end and a proximal end attached to the catheter hub by a bushing, the catheter hub comprising a hub body having an interior surface defining an interior cavity and a proximal opening; positioning a valve proximal of the bushing, the valve comprising an interior surface defining an interior cavity, distal opening, at least one slit, and a plurality of flaps at a proximal end of the valve, and a plurality of interior bumps with at least one interior bump located on each of the plurality of flaps in the interior cavity of the valve; positioning a valve opener inside the interior cavity of the catheter hub and proximal of the valve in a pre-activation position, the valve opener being distally slidable when pushed by a male medical implement into the valve to deflect the plurality of flaps in a distal direction to compress the plurality of interior bumps against the interior surface of the valve, creating an internal reaction force configured to return the flaps to a closed position upon proximal movement of the valve opener to the pre-activation position; placing a needle attached to a needle hub through the catheter hub, the valve, and the catheter tube so that a tip of the needle extends out the open distal end of the catheter tube in a ready to use position; and placing a needle guard inside the interior cavity of the catheter hub and over the needle.

The valve opener may comprise a nose section with an actuation end. The actuation end may contact the valve in the ready to use position.

The valve opener may comprise a nose section with an actuation end. The actuation end may be spaced from the valve in the ready to use position.

A catheter assembly may more broadly be referred to as a needle assembly or a needle device. The catheter assembly may comprise a catheter hub with a catheter tube attached to the hub body via a bushing and a cannula hub or a needle hub with a needle extending through the catheter hub and the catheter tube with the needle tip extending out a distal end or distal opening of the catheter tube in a ready to use position.

In the ready position, the catheter assembly is ready for use, such as to perform a venipuncture or intravenous access. Sometimes the ready position may first require removing a protective cap from the catheter assembly or needle assembly.

Interiorly of the catheter hub, a septum or valve, an actuator or valve opener and a safety clip, such as a needle guard or tip protector, may be provided. The needle can be inserted through a proximal opening of the catheter hub with the needle tip protruding from the distal opening of the catheter tube in a ready to use position.

The proximal opening of the catheter hub can be sized with a female Luer taper, optionally with external threads, to engage with a male Luer tip in a Luer slip or a Luer lock. In use following successful venipuncture an upon the engagement of the male Luer tip, the valve opener may be pushed into the valve to open slit(s) parting a plurality of flaps on the valve to open the valve. The flaps are deflected radially and distally, creating an internal reaction force within the plurality of flaps.

When the valve is in an open position, a fluid path between the male Luer tip and the distal opening of the catheter tube for fluid flow is created. The internal reaction force facilitates the return of the plurality of flaps back to a closed position after the distally directed force on the valve opener is removed from the valve, such as after removal of the male Luer tip.

In the closed position, the valve may create a seal that prevents or limits proximal fluid flow, such as blood blow, across the valve and out the proximal opening of the catheter hub. Following closing of the valve, the same or a different male Luer tip may engage with the catheter hub and push the valve opener in the distal direction to reopen the valve. Thus, the catheter assembly may provide for multiple access blood control.

A flash back plug can be provided at the proximal end of the needle hub, which allows air to vent but stops blood from spilling out the proximal end of the plug when blood enters the flashback chamber during primary flashback. Alternatively, a syringe can be attached to the proximal end of the needle hub. The needle hub further comprises a shoulder or other surfaces that physically contact the catheter hub, such as the proximal end surface of the catheter hub, to axially register the two hubs to set the length of the needle tip projecting out of the distal opening of the catheter tube.

A protective cap with a sleeve and a saddle can be provided to cover the needle during packaging and before use, which is conventional. The saddle can surround at least part of the catheter hub and the needle hub and be removably engaged to the needle hub.

The catheter assembly or the needle assembly may be in a ready position, a transition stage, and a removal stage, where the needle is completely removed from the catheter hub and the tip protector covers the needle tip in a protective position.

During removal of the needle, the tip protector may be activated. The tip protector may embody any number of prior art guards configured for blocking the needle tip of the needle. The tip protector can embody one of the guards shown in U.S. Pat. No. 6,616,630, the contents of which are expressly incorporated herein by reference.

For example, the needle guard can have a proximal wall and two resilient arms and wherein a change in profile on the needle, such as a crimp or a bulge, engages a perimeter defining an opening on the proximal wall of the needle guard to retract the needle guard in the proximal direction out of the catheter hub following successful venipuncture.

The two arms can intersect as described in U.S. Pat. No. 6,616,630. The needle guard arms may be spread by the shaft of the needle in a ready position and engage a guard engagement section(s) of the valve opener and/or of the interior of the catheter hub to retain the needle guard within the catheter in the ready to use position.

In an example, only part of the tip protector or needle guard can extend into one the valve opener while the proximal section of the needle guard, such as the proximal wall, can extend proximally or be located proximally of the proximal most surfaces of the valve opener.

In other examples, one of the needle guards described in the '630 patent may be used with the present catheter assembly. For example, one of the needle guards with only one arm, which runs along a side the shaft of the needle, instead of crossing the needle, may be utilized. Likewise, a distal wall of the one arm may be located to a side of the shaft of the needle in the ready position. When the needle tip is moved proximal of the distal wall, the distal wall can move distal of the needle tip to block accidental contact with the needle tip, and, at the same time, the engagement between the needle guard and the valve opener or between the needle guard and the engagement surface of the interior of the catheter hub is released.

In advancing from the transition stage to the removal stage, the needle tip moves proximally of two distal walls one on each end of the resilient arms of the needle guard, respectively.

Alternatively, the needle guard can have one distal wall and/or one arm. As the two distal walls and hence the two resilient arms are no longer biased outwardly by the needle, for the embodiment in which the two distal walls are biased directly by the needle shaft, the two arms move radially to disengage from the guard engagement section(s) of the valve opener, such as the two bridges of the valve opener, and/or from engagement surfaces formed in the interior of the catheter hub. As the needle continues to move in the proximal direction and the change in profile on the needle engages a perimeter on the proximal wall of the needle guard, the needle guard is moved proximally with the needle to the removal stage position.

Alternatively, the needle guard can clamp onto the shaft of the needle and be removed from the catheter hub as a unit without a change in profile. Notably, in the protective position in which the needle guard covers the needle tip, the valve remains inside interior cavity of the catheter hub. Thus, the valve is located inside the catheter hub in both the ready position of the needle and the protective position of the needle.

The valve may be located inside the catheter hub, or said differently, in the interior cavity of the catheter hub just distal of a nose section of the valve opener. The valve may have a valve body having an outer circumference with a diameter and a length measured orthogonal to the diameter. The valve body may have an elongated portion. The elongated portion may be cylindrical. In some embodiments, the elongated portion of the valve body may be sloped such that the valve forms a frusto-conical structure. The valve may be made with any known elastomer material with those materials that have been certified for medical use being preferred. In some embodiments, the valve may be made from a poly-isoprene material. In some embodiments, the valve may be made from any suitable bio-compatible elastic material. The valve body may have a distal end and a proximal end. An intersection between an end surface at the distal end and the elongated surface of the valve body may form a filleted, chamfered, or straight edge. The valve body may be positioned in a recessed section formed in the interior cavity of the catheter hub, which prevents the valve from axially moving once situated inside the catheter hub.

In some examples, a retainer or holding ring made of a more rigid material than the valve may be incorporated to retain the valve inside the catheter hub. For example, the retainer or holding ring may snap into a shoulder, lip, or groove to secure the valve inside the interior of the catheter hub.

In an example, the recessed section of the interior cavity has a distal shoulder and a proximal shoulder defining a groove therebetween. The valve body length may be sized and shaped to fit within the groove. In some examples, there may be a slight interference between the valve body and the space defined by the proximal and distal shoulders to retain the valve. The proximal end of the valve may contact the proximal shoulder while the distal end may contact the distal shoulder, optionally with a slight compression of the shoulders against the valve body. Viewed differently, the valve can contact the distal shoulder and the proximal shoulder of the recessed section and be restrained thereby in the ready to use position so that the valve body is axially fixed or not axially movable.

In an example, the outer circumference of the valve may form a snug fit or size-on-size fit with the recessed section of the interior cavity of the catheter hub. In other examples, a slight inference fit is provided between the two. In still other examples, a small clearance is provided between the outer circumference of the valve and the interior surface at the recessed section with the proximal end of the valve in contact with the proximal shoulder and the distal end in contact with the distal shoulder of the recessed section. The outer circumference may form a seal with the interior of the catheter hub, at the recessed section. A distal cavity chamber may be provided distal of the distal end of the valve and proximal of the bushing. The distal cavity chamber can have wall surfaces defining the chamber. The wall surfaces can include a tapered section and can include a maximum internal diameter that is smaller than the internal diameter at the recessed section.

The proximal end of the valve comprises a wall structure. The distal end of the valve body may be without a wall structure. The wall structure at the proximal end closes off or serves to obstruct the bore through the valve body at the proximal end of the valve body.

At least one slit is formed through the thickness of the wall structure, measured between a distally facing wall surface and a proximally facing wall surface. If one slit is incorporated, it defines two flaps. In some examples, a plurality of slits are incorporated defining a plurality of flaps. There may be three slits defining three flaps. The three slits may originate from a centrally located point and extend radially from about the centrally located point of the body of the valve, similar to a three-point star, to form the three flaps that can deflect along the slits. In some embodiments, there may be four or more slits defining four or more flaps, respectively.

The two or more flaps may each have an exterior bump extending out therefrom. The exterior bumps can extend axially in the proximal direction, from a planar proximally facing surface of the wall structure. In some embodiments, each flap may have more than one exterior bumps extending out therefrom, such as two or more spaced apart exterior bumps.

There may be three exterior bumps, one on each flap. The exterior bumps may be made as part of the valve. In some embodiments, the exterior bumps may be fastened to the flaps, for example, via a medical-grade adhesive. The exterior bumps may be made from the same material as the valve or another elastic polymer such as rubber or silicon. More preferably, the exterior bumps are integrally molded with the flaps. The exterior bumps extend the reach of the valve in the proximal direction so that a relatively shorter male Luer tip, such as when a male Luer tip having a minimum length is connected to the catheter hub to advance the valve opener. Thus, compared to a valve with the same overall length but without exterior bumps on the proximally facing surface of the wall structure, the present valve has surfaces that extend further in the proximal direction so that the valve opener can abut the valve to open the valve flaps without the valve opener having to undergo the same distal advancement by a standard male Luer tip. Said differently, the male Luer tip can advance the valve opener into the exterior bumps to open the valve flaps without having to abut flat planar surfaces of the wall structure to open the valve flaps. Hence, the exterior bumps, which extend outwardly in the proximal direction from the proximally facing surface of the wall structure, may allow the valve to be opened when the valve opener is advanced towards the valve for a distance less than otherwise required to create an opening between or among the at least one slit. The exterior bumps may have an arcuate shape with rounded edges resembling radially cut segments of a donut, or segments of a torus. In some embodiments, the exterior bumps may each have a different shape. By example and not limitation, some of these shapes may have circular, square, rectangular, triangular, or irregular shaped cross-sections. In some examples, the exterior bumps can be omitted from the flaps, or there can be fewer exterior bumps than the number of flaps.

In an example, the flaps may each have an interior bump extending in the distal direction from the distally facing wall surface inside the interior cavity of the valve. In some embodiments, each flap may have a plurality of interior bumps extending in the distal direction from the distally facing wall surface. There may be three interior bumps, one on each flap. In some embodiments, there may be as many interior bumps as exterior bumps. In some embodiments, the number of interior and exterior bumps are different from one another. In some embodiments, the interior bumps and the exterior bumps may each be made as a single-bodied piece. In some embodiments, the interior bumps and the exterior bumps may be aligned along the proximal end. In some embodiments, the interior bumps and the exterior bumps may be offset along the proximal end.

The interior bumps may be made as part of the valve. In some embodiments, the interior bumps may be fastened to the flaps, for example, via a medical-grade adhesive. The interior bumps may be made from the same material as the valve and/or the exterior bumps. The interior bumps may have the same shape as the exterior bumps. In some embodiments, the interior bumps may have a different shape than the exterior bumps. In some embodiments, each or some of the interior bumps may have a different shape than others.

The interior bumps may make contact with an interior wall that defines part of the interior cavity of the valve when the flaps of the valve are opened by the valve opener, which is advanced in the distal direction by a male Luer tip. The size of the interior bumps and the geometry of the interior wall can be selected to control the amount of compression of the flaps and the interior bumps against the interior wall when the flaps are deflected by the valve opener. This in turn will produce a restoring force for the flaps to return to their closed position upon removal of the male Luer tip. Thus, the interior bumps can be thought of or considered as energizers, which help the flaps to return to their closed position to close the valve and limit or restrict flow across the valve. The energizers can help to prevent or delay plastic deformation and thermal setting, which can affect the flaps' ability to return to their closed state, thereby increasing the longevity and reusability of the valve.

The interior cavity may be defined by a generally cylindrical wall section and a tapered wall extending from the generally cylindrical wall section. In an example, the valve body may terminate at the distal end of the tapered wall. In other embodiments, a bore section may extend from the tapered wall. The bore section can define an opening or passage extending into the interior cavity, from the distal end of the valve. The wall may extend at an angle in the distal direction from the generally cylindrical wall section. In some embodiments, the wall may be parallel to the distal end, and not be tapered. In some embodiments, the wall may have multiple tapers or angles, such as having one or more inflection lines or curves.

In some embodiments, the wall may have a curve, rather than a single slope taper, concaving in a distal direction. In some embodiments, the wall may be eliminated, and the generally cylindrical wall section extends further distally. For an embodiment without the tapered wall, the interior bumps may be sized and shaped to directly contact the interior surface of the valve when the flaps are deflected by the valve opener.

The tapered wall has a minimum diameter along its distal side or end. The minimum diameter defines an opening that may be sized and shaped to accommodate the needle. The bore section, when incorporated distal of the wall, is coincident with the minimum diameter of the wall, which can be circular. The bore section can be generally cylindrical and can be sized and shaped to accommodate the needle. In other examples, the bore section can be non-circular, such as triangular, square, or polygonal in cross-section, with five or more sides. When incorporated, the bore section can be considered a generally cylindrical distal section and the interior surface the generally cylindrical proximal section.

The valve opener may have a nose section that can advance and actuate the flaps of the valve. The nose section may be an elongated structure and can be generally cylindrical or have a draft angle or taper that terminates in an actuation end. The nose section may have a wall surface with a continuous circumference or continuous perimeter section, without a gap or slit, such as a cylinder with a continuous wall. The actuation end of the nose section may have a blunt distal end surface or a sharp edge. In a ready to use position, the actuation end of the valve opener may be spaced away from the proximal end of the valve, including the exterior bumps. In alternative embodiments, the actuation end may contact the valve, such as contact the exterior bumps, but not materially defect the flaps.

The nose section may be sized and shaped so that the distal actuation end contacts the exterior bumps evenly when advancing the flaps. In some examples, the valve opener can incorporate fins or guiding ribs on an exterior that interact with corresponding channels or tracks formed on or in the catheter hub to ensure displacement with little or without unwanted rotation. This may allow for a streamlined flow or more of the fluid to flow from the valve opener through the passage at the deflected flap of the valve. The nose section has a bore. The bore of the nose section and the bore of the valve may have diameters of equal size. In some examples, the bore of the valve opener is larger than the bore of the valve. Exemplary valve openers are disclosed in US Pub. No. 2018/0214682 A1, the contents of which are expressly incorporated herein by reference.

In some embodiments, a plurality of spaced apart slits and/or openings can be provided on the nose section to permit flow or fluid flushing through the spaced apart slits. In some embodiments, when the nose section advances and actuates the flaps, the actuation end deflects the flaps in the distal direction to open the valve, without pushing the interior bumps against the interior wall. In such embodiments, the male Luer tip pushing the valve opener in the distal direction may have a minimum requisite length yet is still capable of opening the valve. As previously described, the exterior projects project axially in the proximal direction to take up some of the axial length. In other embodiments, when the actuation end advances and actuates the flaps, the nose section may push the interior bumps against or into contact with the interior wall. In such embodiments, the male Luer tip may have a length that is longer than the minimum requisite length, up to some maximum length that the male Luer tip can have. Thus, the catheter assembly is configured to operate with male Luer tips of different tip lengths.

Thus, aspects of the invention is understood to include a catheter assembly or needle assembly comprising a valve and a valve opener, and wherein the valve comprises a valve body comprising an elongated section having a distal end and a proximal end, a wall structure at the proximal end, and wherein exterior bumps extend proximally from a proximally facing surface of the wall structure and interior bumps extend distally from a distally facing surface of the wall structure. The wall structure can have a plurality of slits forming a plurality of flaps. Each flap can have at least one exterior bump. Each flap can have at least one interior bump. The flaps can deflect by the valve opener, which can move distally by a male Luer tip inserted into a proximal opening of a catheter hub.

Two actuating elements or plunger elements can extend proximally of the nose section. For example, the two plunger elements can be unitarily formed with the nose section and can extend from the nose section in the proximal direction. A gap or space can be provided between the two plunger elements for positioning the needle guard or tip protector therebetween. In other words, the two plunger elements or actuating elements each comprises at least two lengthwise edges and the edges are spaced from one another. The lengthwise edges of the plunger elements can align, such as being parallel, with a lengthwise axis of the valve opener.

In an example, the actuator or valve opener has a lengthwise axis, the one or more actuating elements extend axially or parallel to the lengthwise axis. In a particular example, two actuating elements are diametrically opposed to one another along the lengthwise axis. As shown, the two actuating elements define an outer diameter having a dimension that is equal to the diameter of the nose section.

In an example, the actuating elements are flexible and deflectable so that when pushed by a male Luer tip, the actuating elements defect or flex. The actuating elements are deflectable by selecting a material that has the requisite resilient properties. In other examples, the actuating elements are deflectable by incorporating one or more weakened sections, such as by incorporating a structurally thin section, by incorporating cut-outs, by employing a small cross-section compared to other sections of the same elongated actuating element, or combinations thereof. Alternatively, the actuating elements can be flexible and deflectable by selecting a material that has the requisite resilient properties and by incorporating one or more weakened sections.

In still other examples, each actuating element has more than one different cross-sectional profile or contour along a length section. For example, an elongated plunger element can have a square profile located adjacent to a crescent-shaped profile.

In an example, the actuating elements are rigid and not deflectable or deformable when loaded, such as when pushed by a male Luer tip. Further, stabilizing elements may be incorporated to increase the rigidity of the two actuating elements. The two actuating elements may each include a cross-sectional profile, at least at a proximal end, that overlaps a push end of the male Luer tip so that the male Luer tip can push the valve actuator into the valve.

The nose section of the valve opener may be configured to engage the valve to open the valve when an axial force is applied by a male Luer tip to the actuating elements towards the distal end of the catheter assembly, such as during the insertion of an IV drip line male Luer connector. Generally, the nose section may be rigid relative to the more pliable valve, which allows the nose section, and more specifically the actuation end, to actuate the valve, such as to deflect the one or more flaps and open the one or more slits on the valve. The nose section may be made of a non-compressible material, such as metal, a rigid plastic, or a hard elastomer for pushing against and opening the valve.

The illustrated valve opener may include a pair of opposed bands or stabilizers connecting the two actuating elements at a location along the length of the actuating elements that are between the nose section and the proximal end of the actuating elements. In some examples, the stabilizers can be located at the proximal end of the two actuating elements so that proximal edges of the stabilizers are generally flush with the proximal end surfaces of the actuating elements. The two stabilizer elements can be referred to as a first or upper stabilizer element and a second or lower stabilizer element.

In one embodiment, the stabilizers or stabilizer elements may be arc-shaped, forming an arc following the interior profile of the catheter hub and connecting one actuating element to another actuating element. The stabilizers or stabilizer elements may form a substantially cylindrical section on the valve opener which may be spaced apart from the nose section of the valve opener. In other words, the valve opener may be elongated and have sections that are continuous along a radial direction and sections with reliefs or through passages through the exterior of the valve opener that are not continuous along the radial direction.

In an example, the stabilizers define a continuous body section along a perimeter or radial direction of the valve opener that is spaced from the nose section, which is also continuous along a perimeter or radial direction. The two stabilizers, or stabilizer elements, may be joined together with the two plunger elements to form a ring structure. The two stabilizer elements may be slightly offset and angled from each other. In some embodiments, there may be one, three, or a different number of actuating elements or stabilizers. In an example, the valve actuator, with the stabilizers or stabilizer elements, is made from plastic, such as by plastic injection molding.

The stabilizers can help the valve actuator remain centered within the catheter hub while the actuator moves, such as when pushed by a male Luer tip. By staying centered, the nose section can be better aligned with the valve, such as the slits, allowing for smooth actuation of the valve. The stabilizers can also provide an engagement, via friction, with the interior cavity of the catheter hub to prevent the actuator from sliding in the proximal direction following removal of the male Luer tip.

A relief, opening, or through passage is provided between the nose section, and each of the two stabilizers. The two reliefs or through passages provide clearance so that the interior or central part of the valve actuator and the interior surface of the catheter hub can be in open communication. In other words, between the continuous section of the nose section and the continuous perimeter section defined by the two stabilizers and the plunger elements, call a stabilizing ring or stabilizing ring, are one or two reliefs, through passages, or openings.

The stabilizing ring of the valve opener can have an inside diameter that is smaller than the diameter defined by the diagonal section or elbows of the two arms of the needle guard when the two arms are biased outwardly by the side of the needle. Thus, during installation of the needle guard into the holding space of the valve opener, the diagonal section of the needle guard can deflect to pass through the stabilizing ring and into the open areas defined by the reliefs.

When the needle guard is positioned between the two plunger elements, the two distal walls of the needle guard, more specifically the two diagonal sections, may be located in the reliefs as discussed above to engage the guard engagement surface on the interior surface of the catheter hub. This allows the needle guard to project from the holding space of the valve opener through the two reliefs to engage with the guard engagement surface of the catheter hub. The needle guard can therefore be retained within the interior of the catheter hub in the ready to use position and during retraction of the needle following successful venipuncture until the needle tip moves proximal of the two distal walls on the needle guard, at which time the needle guard can close over the needle tip and be removed with the needle.

A second undercut or recessed section proximal of the first recessed section can be provided in the interior cavity of the catheter hub for accommodating the two diagonal sections or elbows. The needle guard can therefore be prevented from sliding in the proximal direction during retraction of the needle following successful venipuncture by a shoulder of the second recessed section or by some other surface feature on the interior of the catheter hub, such as a guard engagement surface on the interior of the catheter hub.

The distal edge of one or both stabilizers can provide the restraining surface to prevent the needle guard from early activation during retraction of the needle, prior to the needle tip moving proximally of the two distal walls. In addition to the distal edge, both stabilizers also may have a proximal edge. The distal edge and the proximal edge may not be parallel to the proximal end of the two plunger elements. Instead, the distal edge and the proximal edge of the upper stabilizer may be parallel to each other and the distal edge and the proximal edge of the lower stabilizer may be parallel to each other, but the corresponding edges of the upper and lower stabilizers may not be parallel to one another. Said differently, the upper stabilizer may have a distal tilt while the lower stabilizer may have a proximal tilt.

In some examples, one or both stabilizer elements can have a slit or a channel, thus dividing that arc-shaped stabilizing element into two segments. Nonetheless, even with a slit on one or both stabilizer elements, the stabilizing ring, which can be a non-continuous ring, similar to a ring with one or more slots formed through the ring, can still provide the restraining surface to prevent the needle guard from early activation during retraction of the needle, prior to the needle tip moving proximally of the two distal walls. The restraining surface can also be referred to as a restrict point, choke gap, or choke point since it provides a rigid structure that prevents the needle guard from moving proximally thereof unless or until the needle guard first activates and collapses radially to reduce its radial profile to then slip proximally of the choke point.

In an example, one or two elbows of the needle guard can be restricted by the choke point from moving in the proximal direction until the one or two elbows of the needle guard deflect to reduce radial profile of the needle guard. In an example, when the radial profile of the needle guard is reduced, the needle guard can slip through the bore defined by the stabilizing ring, from a distal position of the stabilizing ring to a proximal position of the stabilizing ring.

The valve opener can be made from a metal material or from a plastic material. When made from a metal material, the valve opener can be formed by bending or deep draw methods and the arc shape cross section of the actuating element can provide added rigidity when pushed by the male Luer tip. Each actuating element can comprise at least two lengthwise edges and a rib can be provided along one or both lengthwise edges to further add structural rigidity. One or more gaps can be provided between any two actuating elements. The gaps can provide clearance or space for fluid flow flowing thereacross, such as during flushing blood or IV infusion. The gap between the actuating elements can define a holding space to accommodate a needle guard.

In some embodiments, a majority or most if not all of the needle guard fits within the holding space formed by the body of the valve opener, between the two plunger elements, in the ready to use position. This allows the catheter hub to be more compact, as less longitudinal space is needed within the catheter hub to fit both the actuator and the tip protector serially lengthwise or when the two only partially overlap in the axial direction. The proximal wall of the needle guard is generally flush or even with the proximal end surfaces of the two plunger elements.

When the needle guard only engages with the distal edge of the relief or through passage in the valve opener, then no deformity or change of diameter is required on the inside wall of the catheter hub and the needle guard can be placed further proximally in the female Luer taper section while complying with the international Luer standard for conical fittings and the overall length of the catheter hub can be further reduced.

When initially inserting the male medical implement, such as a male Luer tip, into the proximal opening of the catheter hub, the male Luer tip initially contacts the two actuating elements on the valve opener to advance a distally directed force on the two actuating elements to open the valve. The distally directed force moves the valve opener in the distal direction until the geometries of the male Luer tip and the proximal opening of the catheter hub stop further distal advancement of the male Luer tip. In an example, a female Luer taper of the catheter hub and a male Luer taper of the male Luer tip mate and block distal advancement of the male Luer tip further into the opening of the catheter hub. A seal is provided by the Luer engagement to prevent fluid from leaking out the proximal opening of the catheter hub.

As the valve opener moves distally by the distal advancement of the male Luer tip, the nose section of the valve actuator is urged distally and pushes against the proximal end of the valve. In particular, the nose section of the valve opener initially pushes against the proximal end of the valve and since the valve is axially fixed within the first recessed section of the catheter hub, the one or more flaps on the valve deflect radially and in the distal direction. A reversible or internal force may be created within the valve upon the one or more flaps being pushed. Fluid from the male Luer tip can then flow through the catheter hub, through the valve, and through the lumen of the catheter tube.

Alternatively, a suction can be applied by the male medical instrument, such as a syringe or vacuum blood collection tube, and blood aspirated from the patient. This is often done for testing samples before infusion therapy is commenced. Also, typically any remaining blood is first flushed from the inside of the catheter hub before infusion therapy is commenced.

After removal of the male Luer tip, the valve opener may disengage from the valve. The flaps may then return to their un-deformed position due to the internal force initially created by opening of the valve. Hence, the valve may be used or actuated multiple times, the valve opener and valve returning to the pre-activation position when the male tip is removed.

The nose section may be configured so that when pushed into the valve during activation, the actuation or activation end does not extend distally past the flaps of the valve. This configuration can ensure that the valve opener is pushed back by the flaps when the male Luer tip is removed. The distal movement of the male Luer tip is a minimum distal movement. In other embodiments, there may be a maximum distal movement. The difference between the maximum movement and minimum movement of a standard Luer connector may be approximately 2.5 millimeters.

Methods of making and of using the catheter assemblies and their components described elsewhere herein are within the scope of the present disclosure.

### BRIEF DESCRIPTION OF THE DRAWINGS

These and other features and advantages of the present device, system, and method will become appreciated as the same becomes better understood with reference to the specification, claims and appended drawings wherein:

FIG. 1 is a schematic cross-sectional side view of a catheter assembly in a ready position in which the needle tip extends out a distal end of a catheter tube.

FIG. 2 is a schematic cross-sectional side view of the catheter assembly of FIG. 1 in a transition position or state in which the needle is in the process of being removed from the catheter tube and the catheter hub, such as following successful venipuncture.

FIG. 3 is a schematic cross-sectional side view of the catheter assembly of FIG. 1 in which the needle has completely separated from the catheter hub and the needle tip is covered by a needle guard.

FIG. 4 is a schematic cross-sectional side view of the catheter assembly of FIG. 1 in which the catheter hub is connected with a male Luer tip and a valve opener is advanced for a minimum distance by the male Luer tip to open a valve.

FIG. 5 is a schematic cross-sectional side view of the catheter assembly of FIG. 1 in which the catheter hub is connected with a male Luer tip and the valve opener is advanced for a maximum distance by the male Luer tip into the valve.

FIG. 6 shows a perspective view of the valve of the catheter assembly of FIG. 1.

FIG. 7 shows a cross-sectional perspective view of the valve of the catheter assembly of FIG. 1.

FIG. 8 shows a front, or distal end, view of the valve of the catheter assembly of FIG. 1.

FIG. 9 shows a side view of the valve of the catheter assembly of FIG. 1.

FIG. 10 shows a rear, or proximal end, view of the valve of the catheter assembly of FIG. 1.

FIG. 11 shows a side cross-sectional view of the valve of the catheter assembly of FIG. 1.

FIG. 12 shows a rear cross-sectional view of the valve of the catheter assembly of FIG. 1.

### DETAILED DESCRIPTION

The detailed description set forth below in connection with the appended drawings is intended as a description of the presently preferred embodiments of catheter assemblies with control valves provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

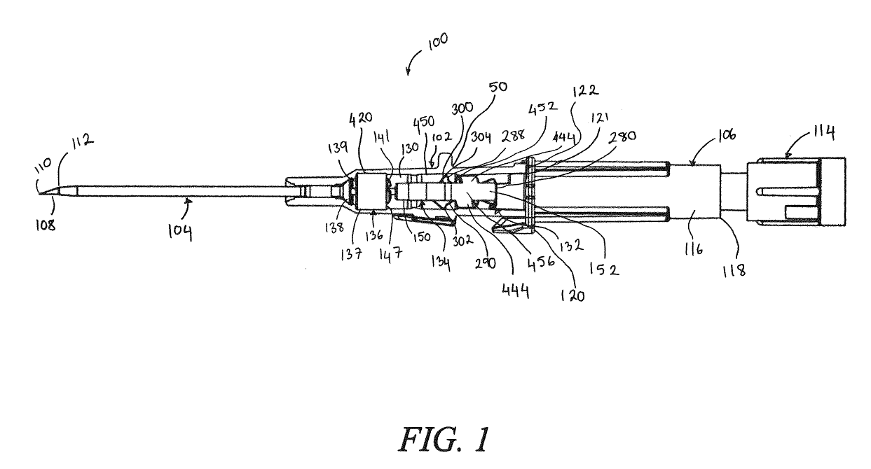

With reference now to FIG. 1, a catheter assembly 100, which may more broadly be referred to as a needle assembly or a needle device, is shown comprising a catheter hub 102 with a catheter tube 104 attached to the hub body 50 via a bushing 138 and a cannula hub or a needle hub 106 with a needle 108 extending through the catheter hub 102 and the catheter tube 104 with the needle tip 110 extending out a distal end or distal opening 112 of the catheter tube in a ready to use position. In the ready position, the catheter assembly 100 is ready for use, such as to perform a venipuncture or intravenous access. Sometimes the ready position may first require removing a protective cap (not shown) from the catheter assembly or needle assembly 100.

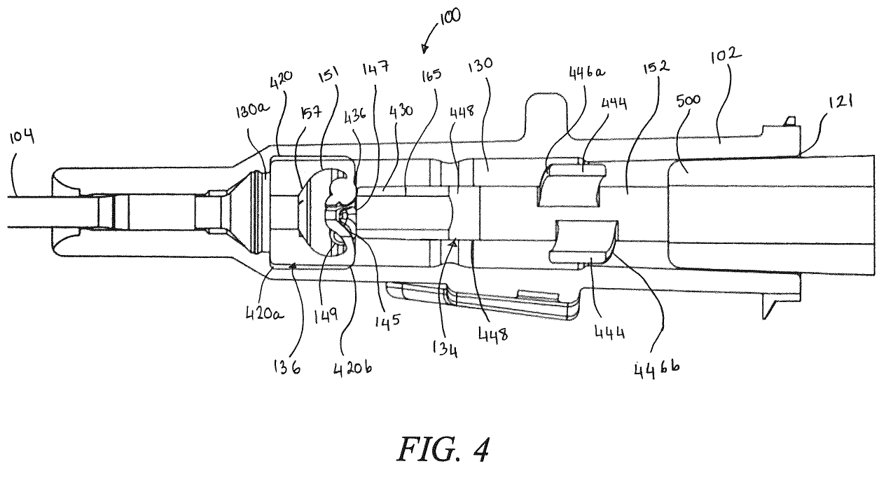

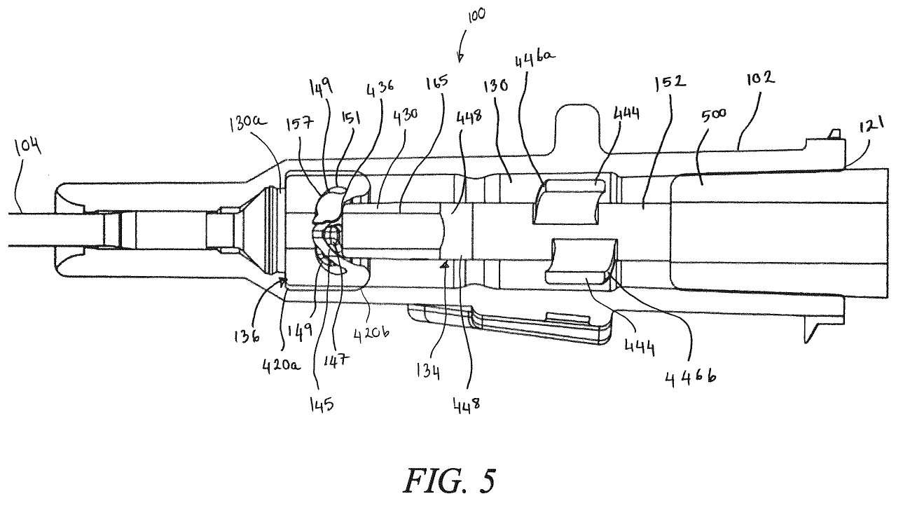

Interiorly of the catheter hub 102, a septum or valve 136, an actuator or valve opener 134 and a safety clip 132, such as a needle guard or tip protector, may be provided. The needle 108, can be inserted through a proximal opening 121 of the catheter hub 102 with the needle tip protruding from the distal opening 112 of the catheter tube 104 in a ready to use position. The proximal opening 121 of the catheter hub 102 can be sized with a female Luer taper, optionally with external threads, to engage a male Luer tip 500 (see FIGS. 4-5) in a Luer slip or a Luer lock.

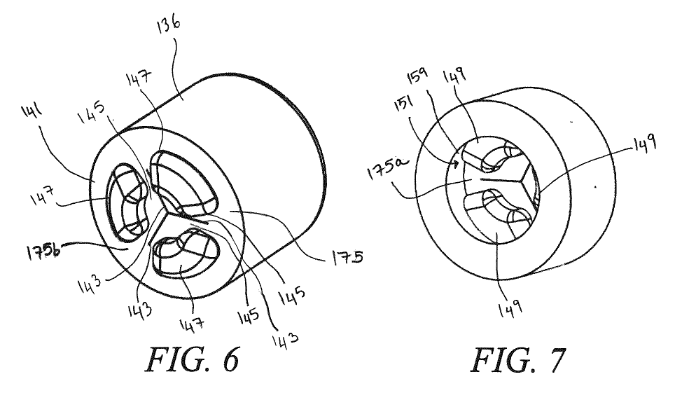

In use following successful venipuncture and upon the engagement of the male Luer tip 500, the valve opener 134 is pushed into the valve 136 to open one or more slits 143 (see FIGS. 6 and 10) parting a plurality of flaps 145 (see FIGS. 4-6, 10) on the valve 136 to open the valve 136. The flaps 145 are deflected radially and distally, creating an internal reaction force within the plurality of flaps 145. When the valve 136 is in an open position (see FIGS. 4-5), a fluid path between the male Luer tip 500 and the distal opening 112 of the catheter tube 104 for fluid flow is created. The internal reaction force facilitates the return of the plurality of flaps 145 back to a closed position (see FIGS. 6-11) after the distally directed force on the valve opener 134 is removed, such as after removal of the male Luer tip 500. In the closed position, the valve 136 may create a seal that prevents or limits proximal fluid flow, such as blood blow, across the valve and out the proximal opening 121 of the catheter hub 102. Following closing of the valve 136, the same or a different male Luer tip 500 may engage with the catheter hub 102 and push the valve opener 134 in the distal direction to reopen the valve 136. Thus, the catheter assembly 100 may provide for multiple access blood control.

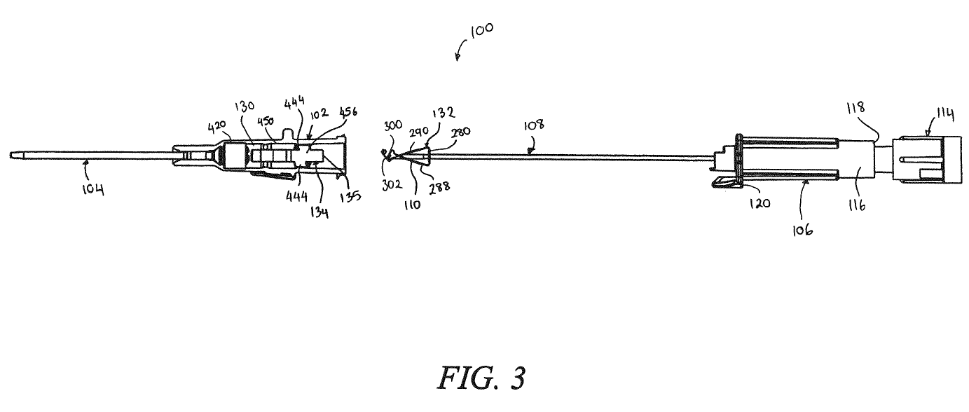

A flash back plug 114 (see FIGS. 1 and 3) can be provided at the proximal end 118 of the needle hub 106, which allows air to vent but stops blood from spilling out the proximal end of the plug when blood enters the flashback chamber 116 during primary flashback. Alternatively, a syringe (not shown) can be attached to the proximal end 118 of the needle hub 106. The needle hub 106 further comprises a shoulder 120 or other surfaces that physically contact the catheter hub 102, such as the proximal end surface 122 of the catheter hub, to axially register the two hubs 102, 106 to set the length of the needle tip 110 (see FIGS. 1 and 3) projecting out of the distal opening 112 of the catheter tube 104.

A protective cap (not shown) with a sleeve and a saddle can be provided to cover the needle 108 during packaging and before use, which is conventional. The saddle can surround at least part of the catheter hub 102 and the needle hub 106 and be removably engaged to the needle hub 106.

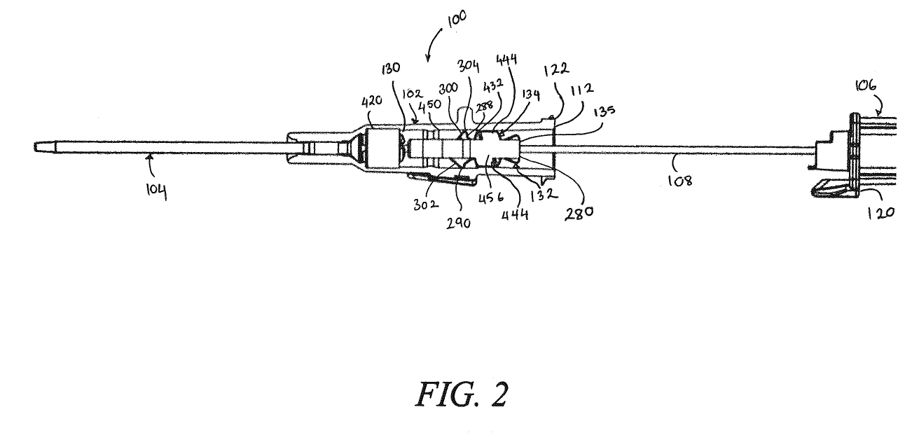

With general reference to FIGS. 1-3, stages of removal of the needle 108 from the catheter tube 104 and the catheter hub 102 are shown, such as during retraction following successful venipuncture. FIG. 1 shows the ready position of the needle assembly, FIG. 2 shows a transition stage, and FIG. 3 shows a complete removal stage of the needle 108 from the catheter hub 102 and the tip protector 132 covering the needle tip 110 in a protective position. During removal of the needle 108, the tip protector 132 may be activated. The tip protector 132 may embody any number of prior art guards configured for blocking the needle tip 110 of the needle. In the exemplary embodiment shown, the tip protector 132 can embody one of the guards shown in U.S. Pat. No. 6,616,630, the contents of which are expressly incorporated herein by reference. For example, the needle guard 132 can have a proximal wall 280 and two resilient arms 288, 290 and wherein a change in profile 144 (see FIG. 3) on the needle 108, such as a crimp or a bulge, engages a perimeter defining an opening on the proximal wall 280 of the needle guard 132 to retract the needle guard 132 in the proximal direction out of the catheter hub 102 following successful venipuncture. The two arms 288, 290 can intersect as described in U.S. Pat. No. 6,616,630 and shown in FIG. 3

The needle guard arms 288, 290 may be spread by the shaft of the needle 108 in a ready position and engage a guard engagement section(s) (not shown) of the valve opener 134 and/or of the interior of the catheter hub to retain the needle guard within the catheter in the ready to use position. In an example, only part of the tip protector or needle guard 132 can extend into one the valve opener 134 while the proximal section of the needle guard 132, such as the proximal wall 280, can extend proximally or be located proximally of the proximal most surfaces of the valve opener 134. In other examples, one of the needle guards (not shown) described in the '630 patent may be used with the present catheter assembly. For example, one of the needle guards with only one arm, which runs along a side the shaft of the needle 108 instead of crossing the needle 108, may be utilized. Likewise, a distal wall of the one arm may be located to a side of the shaft of the needle 108 in the ready position. When the needle tip 110 is moved proximal of the distal wall, the distal wall can move distal of the needle tip 110 to block accidental contact with the needle tip 110, and, at the same time, the engagement between the needle guard 132 and the valve opener 134 or between the needle guard 132 and the engagement surface of the interior of the catheter hub is released.

In transitioning from the position of FIG. 2 to the position of FIG. 3, the needle tip 110 moves proximally of two distal walls 300, 302, one on each end of the resilient arms 288, 290 of the needle guard 132, respectively. Alternatively, the needle guard 132 can have one distal wall and/or one arm. As the two distal walls 300, 302 and hence the two resilient arms 288, 290 are no longer biased outwardly by the needle 108, for the embodiment in which the two distal walls are biased directly by the needle shaft, the two arms 288, 290 move radially to disengage from the guard engagement section(s) of the valve opener 134, such as the two bridges of the valve opener, and/or from engagement surfaces formed in the interior of the catheter hub. As the needle continues to move in the proximal direction and the change in profile 144 on the needle (see FIG. 3) engages a perimeter on the proximal wall 280 of the needle guard 132, the needle guard 132 is moved proximally with the needle 108 to the position shown in FIG. 3. Alternatively, the needle guard 132 can clamp onto the shaft of the needle 108 and be removed from the catheter hub 102 as a unit without a change in profile 144. Notably, in the protective position in which the needle guard 132 covers the needle tip 110, the valve 136 remains inside interior cavity 130 of the catheter hub 102. Thus, the valve 136 is located inside the catheter hub 102 in both the ready position of the needle 108 and the protective position of the needle 108. Viewed from another perspective, the valve 136 is located inside the catheter hub 102 in both the ready to use position of the catheter assembly 100 (see FIG. 1), in which the needle tip 110 projects out a distal opening 112 of the catheter tube 104, and a protective position of the catheter assembly (see FIG. 3), in which the needle 108 is removed from the catheter hub 102 and the needle tip 110 is covered by the needle guard 132.

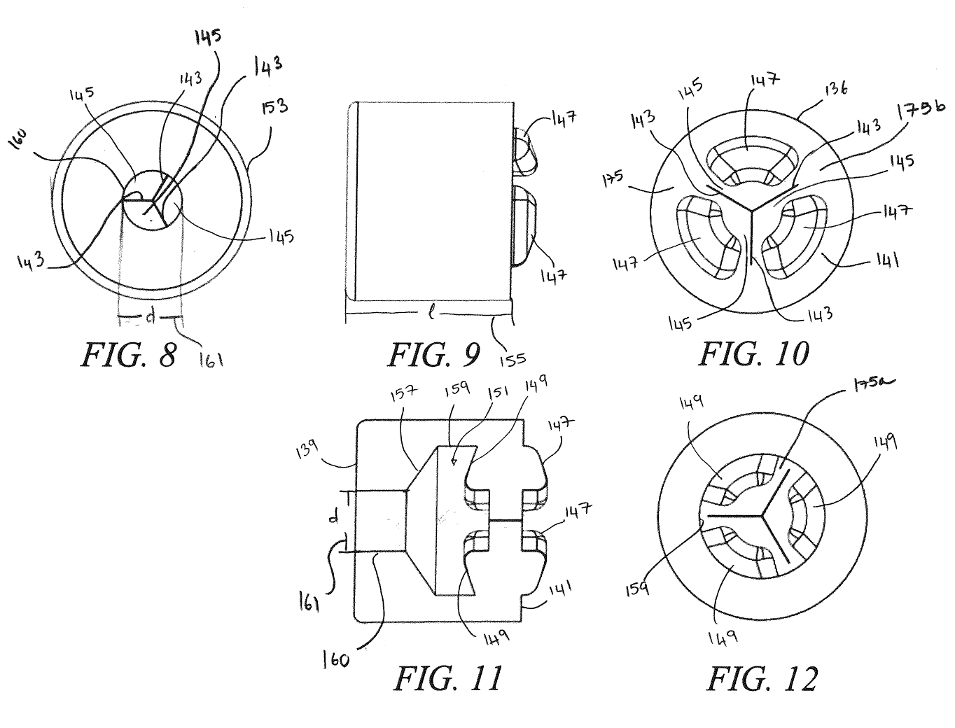

With reference again to FIG. 1, the valve 136 may be located inside the catheter hub 102, or said differently, in the interior cavity 130 of the catheter hub 102 just distal of a nose section 150 of the valve opener 134. The valve 136 may have a valve body 137 having an outer circumference 153 with a diameter (see FIG. 8) and a length 155 (see FIG. 9) measured orthogonal to the diameter. The valve body 137 may have an elongated portion. The elongated portion may be cylindrical. In some embodiments, the elongated portion of the valve body 137 may be sloped such that the valve 136 forms a frusto-conical structure. The valve 136 may be made with any known elastomer material with those materials that have been certified for medical use being preferred. In some embodiments, the valve may be made from a poly-isoprene material. In some embodiments, the valve may be made from any suitable bio-compatible elastic material.

The valve body 137 may have a distal end 139 and a proximal end 141. An intersection between an end surface at the distal end 139 and the elongated surface of the valve body 137 may form a filleted (see FIG. 9), chamfered, or straight edge. The valve body 137 may be positioned in a recessed section 420 formed in the interior cavity 130 of the catheter hub 102, which prevents the valve 136 from axially moving once situated inside the catheter hub 102. In some examples, a retainer or holding ring made of a more rigid material than the valve may be incorporated to retain the valve inside the catheter hub. For example, the retainer or holding ring may snap into a shoulder, lip or groove to secure the valve inside the interior of the catheter hub.

In an example, the recessed section 420 of the interior cavity 130 has a distal shoulder 420a and a proximal shoulder 420b (see FIGS. 4-5) defining a groove therebetween. The valve body length 155 (see FIG. 9) may be sized and shaped to fit within the groove. In some examples, there may be a slight interference between the valve body and the space defined by the proximal and distal shoulders to retain the valve. The proximal end 141 of the valve 136 may contact the proximal shoulder 420b while the distal end 139 may contact the distal shoulder 420a, optionally with a slight compression of the shoulders against the valve body. Viewed differently, the valve 136 can contact the distal shoulder 420a and the proximal shoulder 420b of the recessed section 420 and be restrained thereby in the ready to use position so that the valve body 137 is axially fixed or not axially movable.

In an example, the outer circumference 153 of the valve 136 (see FIG. 8) may form a snug fit or size-on-size fit with the recessed section 420 of the interior cavity 130 of the catheter hub 102. In other examples, a slight inference fit is provided between the two. In still other examples, a small clearance is provided between the outer circumference 153 of the valve 136 and the interior surface at the recessed section 420 with the proximal end 141 of the valve 136 in contact with the proximal shoulder 420b and the distal end 139 in contact with the distal shoulder 420a of the recessed section 420. The outer circumference 153 may form a seal with the interior of the catheter hub, at the recessed section. A distal cavity chamber 130a (see FIGS. 4-5) may be provided distal of the distal end 139 of the valve 136 and proximal of the bushing 138. The distal cavity chamber 130a can have wall surfaces defining the chamber. The wall surfaces can include a tapered section and can include a maximum internal diameter that is smaller than the internal diameter at the recessed section 420.

Referring now to FIGS. 6 and 10, a perspective and front view of a valve 136, respectively, are shown with a proximal end 141. In the present embodiment, the proximal end 141 of the valve 136 comprises a wall structure 175. The distal end of the body 137 may be without a wall structure. The wall structure 175 at the proximal end closes off or serves to obstruct the bore through the valve body 137 at the proximal end of the valve body. At least one slit 143 is formed through the thickness of the wall structure 175, measured between a distally facing wall surface 175a (see FIGS. 7 and 12) and a proximally facing wall surface 175b. If one slit 143 is incorporated, it defines two flaps. In some examples, a plurality of slits 143 are incorporated defining a plurality of flaps 145. As shown in FIGS. 6 and 10, there may be three slits 143 defining three flaps 145. The three slits 143 may originate from a centrally located point and extend radially from about the centrally located point of the body 137 of the valve 136, similar to a three-point star, to form the three flaps 145 that can deflect along the slits 143. In some embodiments, there may be four or more slits 143 defining four or more flaps 145, respectively. The two or more flaps 145 may each have an exterior bump 147 extending out therefrom. The exterior bumps 147 can extend axially in the proximal direction, from a planar proximally facing surface 175b of the wall structure 175 (FIG. 10). In some embodiments, each flap 145 may have more than one exterior bumps 147 extending out therefrom, such as two or more spaced apart exterior bumps. As shown in FIGS. 6 and 10, there may be three exterior bumps, one on each flap 145. The exterior bumps 147 may be made as part of the valve 136. (See FIG. 11) In some embodiments, the exterior bumps 147 may be fastened to the flaps 145, for example, via a medical-grade adhesive. The exterior bumps 147 may be made from the same material as the valve 136 or another elastic polymer such as rubber or silicon. Preferably, the exterior bumps 147 are integrally molded with the flaps 145.

The exterior bumps 147 extend the reach of the valve in the proximal direction so that a relatively shorter male Luer tip, such as when a male Luer tip 500 having a minimum length is connected to the catheter hub 102 to advance the valve opener 134 (FIG. 4). Thus, compared to a valve with the same overall length but without exterior bumps 147 on the proximally facing surface of the wall structure 175, the present valve has surfaces that extend further in the proximal direction so that the valve opener 134 (FIG. 1) can abut the valve to open the valve flaps without the valve opener having to undergo the same distal advancement by a standard male Luer tip. Said differently, the male Luer tip can advance the valve opener into the exterior bumps 147 to open the valve flaps without having to abut flat planar surfaces of the wall structure 175 to open the valve flaps. Hence, the exterior bumps 147, which extend outwardly in the proximal direction from the proximally facing surface 175b of the wall structure 175, may allow the valve 136 to be opened when the valve opener 134 is advanced towards the valve 136 for a distance less than otherwise required to create an opening between or among the at least one slit 143. As shown in FIGS. 6, 9, and 10, the exterior bumps 147 may have an arcuate shape with rounded edges resembling radially cut segments of a donut, or segments of a torus. In some embodiments, the exterior bumps 147 may each have a different shape. By example and not limitation, some of these shapes may have circular, square, rectangular, triangular, or irregular shaped cross-sections. In some examples, the exterior bumps can be omitted from the flaps, or there can be fewer exterior bumps than the number of flaps.

Referring now to FIGS. 7 and 11-12, an interior cavity 151 of the valve 136 is shown. In an example, the flaps 145 may each have an interior bump 149 extending in the distal direction from the distally facing wall surface 175a inside the interior cavity 151 of the valve 136. In some embodiments, each flap 145 may have a plurality of interior bumps 149 extending in the distal direction from distally facing wall surface 175a. As shown in FIGS. 7 and 11-12, there may be three interior bumps 149, one on each flap 145. In some embodiments, there may be as many interior bumps 149 as exterior bumps 147. In some embodiments, the number of interior and exterior bumps are different from one another. In some embodiments, the interior bumps 149 and the exterior bumps 147 may each be made as a single-bodied piece. In some embodiments, the interior bumps 149 and the exterior bumps 147 may be aligned along the proximal end 141. In some embodiments, the interior bumps 149 and the exterior bumps 147 may be offset along the proximal end 141. As shown in FIG. 11, the interior bumps 149 may be made as part of the valve 136. In some embodiments, the interior bumps 149 may be fastened to the flaps 145, for example, via a medical-grade adhesive. The interior bumps 149 may be made from the same material as the valve 136 and/or the exterior bumps 147. The interior bumps 149 may have the same shape as the exterior bumps 147, as shown in FIGS. 7 and 12 and described elsewhere herein. In some embodiments, the interior bumps 149 may have a different shape than the exterior bumps 147. In some embodiments, each or some of the interior bumps 149 may have a different shape than others.

The interior bumps 149 may make contact with an interior wall 157 that defines part of the interior cavity 151 of the valve 136 when the flaps 145 of the valve 136 are opened by the valve opener 134, which is advanced in the distal direction by a male Luer tip (FIG. 5). The size of the interior bumps 149 and the geometry of the interior wall 157 can be selected to control the amount of compression of the flaps 145 and the interior bumps 149 against the interior wall 157 when the flaps are deflected by the valve opener. This in turn will produce a restoring force for the flaps to return to their closed position upon removal of the male Luer tip. Thus, the interior bumps 149 can be thought of or considered as energizers, which help the flaps to return to their closed position to close the valve and limit or restrict flow across the valve. The energizers can help to prevent or delay plastic deformation and thermal setting, which can affect the flaps' ability to return to their closed state, thereby increasing the longevity and reusability of the valve 136.

The interior cavity 151 may be defined by a generally cylindrical wall section 159 and a tapered wall 157 extending from the generally cylindrical wall section 159. In an example, the valve body 137 may terminate at the distal end of the tapered wall 157. In other embodiments, a bore section 160 may extend from the tapered wall 157. The bore section 160 can define an opening or passage extending into the interior cavity 151, from the distal end of the valve. The wall 157 may extend at an angle in the distal direction from the generally cylindrical wall section 159, as shown in FIG. 11. In some embodiments, the wall 157 may be parallel to the distal end 139, and not be tapered. In some embodiments, the wall 157 may have multiple tapers or angles, such as having one or more inflection lines or curves.

In some embodiments, the wall 157 may have a curve, rather than a single slope taper as shown, concaving in a distal direction (FIGS. 4-5). In some embodiments, the wall 157 may be eliminated and the generally cylindrical wall section 159 extends further distally. For an embodiment without the tapered wall 157, the interior bumps 149 may be sized and shaped to directly contact the interior surface 159 of the valve 136 when the flaps are deflected by the valve opener. The tapered wall 157 has a minimum diameter 161 along its distal side or end. The minimum diameter 161 defines an opening that may be sized and shaped to accommodate the needle 108 (FIG. 1). As shown in FIG. 8, the bore section 160, when incorporated distal of the wall 157, is coincident with the minimum diameter 161 of the wall 157, which can be circular. The bore section 160 can be generally cylindrical and can be sized and shaped to accommodate the needle 108. In other examples, the bore section 160 can be non-circular, such as triangular, square, or polygonal in cross-section, with five or more sides. When incorporated, the bore section 160 can be considered a generally cylindrical distal section and the interior surface 159 the generally cylindrical proximal section.

With reference now to FIGS. 4-5, the valve opener 134 may have a nose section 430 that can advance and actuate the flaps 145 of the valve 136. The nose section 430 may be an elongated structure and can be generally cylindrical or have a draft angle or taper that terminates in an actuation end 436. The nose section 430 may have a wall surface with a continuous circumference or continuous perimeter section, without a gap or slit, such as a cylinder with a continuous wall. The actuation end 436 of the nose section 430 may have a blunt distal end surface or a sharp edge. In a ready to use position (see FIG. 1), the actuation end 436 of the valve opener 134 may be spaced away from the proximal end 141 of the valve 136, including the exterior bumps 147. In alternative embodiments, the actuation end 436 may contact the valve, such as contact the exterior bumps 147, but not materially defect the flaps.

The nose section 430 may be sized and shaped so that the distal actuation end 436 contacts the exterior bumps 147 evenly when advancing the flaps 145. In some examples, the valve opener 134 can incorporate fins or guiding ribs on an exterior that interact with corresponding channels or tracks formed on or in the catheter hub to ensure displacement with little or without unwanted rotation. This may allow for a streamlined flow or more of the fluid to flow from the valve opener 134 through the passage at the deflected flap of the valve 136. The nose section 430 has a bore 165. The bore 165 of the nose section 430 and the bore 160 of the valve 136 may have diameters of equal size. In some examples, the bore 165 of the valve opener is larger than the bore 160 of the valve. Exemplary valve openers are disclosed in US Pub. No. 2018/0214682 A1, the contents of which are expressly incorporated herein by reference.

In some embodiments, a plurality of spaced apart slits and/or openings can be provided on the nose section 430 to permit flow or fluid flushing through the spaced apart slits. In some embodiments, such as that of FIG. 4, when the nose section 430 advances and actuates the flaps 145, the actuation end 436 deflects the flaps 145 in the distal direction to open the valve, without pushing the interior bumps 149 against the interior wall 157. In such embodiments, the male Luer tip 500 pushing the valve opener 134 in the distal direction may have a minimum requisite length yet is still capable of opening the valve 136. As previously described, the exterior projects 147 project axially in the proximal direction to take up some of the axial length. In other embodiments, such as that of FIG. 5, when the actuation end 436 advances and actuates the flaps 145, the nose section 430 may push the interior bumps 149 against or into contact with the interior wall 157. In such embodiments, the male Luer tip 500 may have a length that is longer than the minimum requisite length, up to some maximum length that the male Luer tip 500 can have, such as the male Luer tip 500 of FIG. 5. Thus, the catheter assembly is configured to operate with male Luer tips of different tip lengths.

Either the interior bumps, the exterior bumps, or both the interior and exterior bumps on the wall structure at the proximal end of the valve, such as on the flaps of the wall structure, can extend the thickness of the wall structure. The thickness can be measured from the exterior most surface at the proximal side to the exterior most surface at the distal side of the wall structure, where the flaps are provided. When extending the thickness of the wall structure, such as the interior bumps, the flaps can be deflected by a valve opener to contact an interior surface of the valve with a male medical implement without having to deflect the flaps the same amount compared to when no bumps are incorporated.

Thus, aspects of the invention are understood to include a catheter assembly or needle assembly comprising a valve and a valve opener, and wherein the valve comprises a valve body comprising an elongated section having a distal end and a proximal end, a wall structure at the proximal end, and wherein exterior bumps extend proximally from a proximally facing surface of the wall structure and interior bumps extend distally from a distally facing surface of the wall structure. The wall structure can have a plurality of slits forming a plurality of flaps. Each flap can have at least one exterior bump. Each flap can have at least one interior bump. The flaps can deflect by the valve opener, which can move distally by a male Luer tip inserted into a proximal opening of a catheter hub.

Two actuating elements or plunger elements 152 can extend proximally of the nose section 430. For example, the two plunger elements 152 can be unitarily formed with the nose section 430 and can extend from the nose section 430 in the proximal direction. A gap or space can be provided between the two plunger elements 152 for positioning the needle guard or tip protector 132 therebetween. In other words, the two plunger elements or actuating elements 152 each comprises at least two lengthwise edges and the edges are spaced from one another. The lengthwise edges of the plunger elements 152 can align, such as being parallel, with a lengthwise axis of the valve opener 134.

In an example, the actuator or valve opener 134 has a lengthwise axis, the one or more actuating elements 152 extend axially or parallel to the lengthwise axis. In a particular example, two actuating elements 152 are diametrically opposed to one another along the lengthwise axis. As shown, the two actuating elements 152 define an outer diameter having a dimension that is equal to the diameter of the nose section 430.

In an example, the actuating elements 152 are flexible and deflectable so that when pushed by a male Luer tip 500, the actuating elements defect or flex. The actuating elements are deflectable by selecting a material that has the requisite resilient properties. In other examples, the actuating elements are deflectable by incorporating one or more weakened sections, such as by incorporating a structurally thin section, by incorporating cut-outs, by employing a small cross-section compared to other sections of the same elongated actuating element, or combinations thereof. Alternatively, the actuating elements 152 can be flexible and deflectable by selecting a material that has the requisite resilient properties and by incorporating one or more weakened sections.

In still other examples, each actuating element 152 has more than one different cross-sectional profile or contour along a length section. For example, an elongated plunger element can have a square profile located adjacent to a crescent-shaped profile.

In an example, the actuating elements 152 are rigid and not deflectable or deformable when loaded, such as when pushed by a male Luer tip 500. Further, stabilizing elements may be incorporated to increase the rigidity of the two actuating elements 152. The two actuating elements 152 may each include a cross-sectional profile, at least at a proximal end, that overlaps a push end of the male Luer tip 500 so that the male Luer tip 500 can push the valve actuator 134 into the valve 136.

The nose section 430 of the valve opener 134 may be configured to engage the valve 136 to open the valve 136 when an axial force is applied by a male Luer tip 500 to the actuating elements 152 towards the distal end of the catheter assembly 100, such as during the insertion of an IV drip line male Luer connector. Generally, the nose section 430 may be rigid relative to the more pliable valve 136, which allows the nose section 430, and more specifically the actuation end 436, to actuate the valve 136, such as to deflect the one or more flaps 145 and open the one or more slits 143 on the valve 136. The nose section 430 may be made of a non-compressible material, such as metal, a rigid plastic, or a hard elastomer for pushing against and opening the valve 136.

The illustrated valve opener 134 in FIGS. 1-5 includes a pair of opposed bands or stabilizers 444 (collectively or individually referred to as stabilizer or stabilizers 444) connecting the two actuating elements 152 at a location along the length of the actuating elements 152 that are between the nose section 430 and the proximal end 135 (see FIG. 3) of the actuating elements 152. In some examples, the stabilizers 444 can be located at the proximal end 135 of the two actuating elements 152 so that proximal edges of the stabilizers 444 are generally flush with the proximal end 135 surfaces of the actuating elements 152. The two stabilizer elements 444 can be referred to as a first or upper stabilizer element and a second or lower stabilizer element.

In one embodiment, the stabilizers or stabilizer elements 444 may be arc-shaped, forming an arc following the interior profile of the catheter hub 102 and connecting one actuating element 152 to another actuating element 152. The stabilizers or stabilizer elements 444 may form a substantially cylindrical section on the valve opener 134 which may be spaced apart from the nose section 430 of the valve opener 134. In other words, the valve opener 134 may be elongated and have sections that are continuous along a radial direction and sections with reliefs or through passages through the exterior of the valve opener 134 that are not continuous along the radial direction.

In an example, the stabilizers 444 define a continuous body section along a perimeter or radial direction of the valve opener 134 that is spaced from the nose section 430, which is also continuous along a perimeter or radial direction. The two stabilizers or stabilizer elements 444, may be joined together with the two plunger elements 152 to form a ring structure. The two stabilizer elements 444 may be slightly offset and angled from each other. In some embodiments, there may be one, three, or a different number of actuating elements 152 or stabilizers 444. In an example, the valve actuator 134, with the stabilizers or stabilizer elements 444, is made from plastic, such as by plastic injection molding.

The stabilizers 444 can help the valve actuator 134 remain centered within the catheter hub 102 while the actuator moves, such as when pushed by a male Luer tip 500. By staying centered, the nose section 430 can be better aligned with the valve 136, such as the slits 143, allowing for smooth actuation of the valve 136. The stabilizers 444 can also provide an engagement, via friction, with the interior cavity 130 of the catheter hub 102 to prevent the actuator 134 from sliding in the proximal direction following removal of the male Luer tip 500.

A relief, opening, or through passage 448 is provided between the nose section 430, and each of the two stabilizers 444. The two reliefs or through passages 448 provide clearance so that the interior or central part of the valve actuator 134 and the interior surface of the catheter hub 102 can be in open communication. In other words, between the continuous section of the nose section 430 and the continuous perimeter section defined by the two stabilizers 444 and the plunger elements 152, call a stabilizing ring or stabilizing ring 456 (see FIGS. 1-3), are one or two reliefs, through passages, or openings 448.

Referring back to FIGS. 1-3, the stabilizing ring 456 of the valve opener 134 can have an inside diameter that is smaller than the diameter defined by the diagonal section or elbows 304 of the two arms 288, 290 of the needle guard 132 when the two arms 288, 290 are biased outwardly by the side of the needle 108. Thus, during installation of the needle guard 132 into the holding space of the valve opener 134, the diagonal section 304 of the needle guard 132 can deflect to pass through the stabilizing ring 456 and into the open areas defined by the reliefs 448.

When the needle guard 132 is positioned between the two plunger elements 152, the two distal walls 300, 302 of the needle guard 132, more specifically the two diagonal sections 304, can be located in the reliefs 448 as discussed above to engage the guard engagement surface (not shown) on the interior surface of the catheter hub 102. This allows the needle guard 132 to project from the holding space of the valve opener 134 through the two reliefs 448 to engage with the guard engagement surface of the catheter hub 102. The needle guard 132 can therefore be retained within the interior of the catheter hub 102 in the ready to use position and during retraction of the needle 108 following successful venipuncture until the needle tip 110 moves proximal of the two distal walls 300, 302 on the needle guard 132, at which time the needle guard 132 can close over the needle tip 110 and be removed with the needle 102 as shown in FIG. 3.

Still referring to FIGS. 1-3, a second undercut or recessed section 450 proximal of the first recessed section 420 can be provided in the interior cavity 130 of the catheter hub 102 for accommodating the two diagonal sections or elbows 304. The needle guard 132 can therefore be prevented from sliding in the proximal direction during retraction of the needle 108 following successful venipuncture by a shoulder 452 of the second recessed section 450 or by some other surface feature on the interior of the catheter hub 102, such as a guard engagement surface (not shown) on the interior of the catheter hub 102. Distal edge 446a (see FIGS. 4-5) of one or both stabilizers 444 can provide the restraining surface to prevent the needle guard 132 from early activation during retraction of the needle 108, prior to the needle tip 110 moving proximally of the two distal walls 300, 302. In addition to the distal edge 446a, both stabilizers 444 also may have a proximal edge 446b (see FIGS. 4-5). The distal edge 446a and the proximal edge 446b may not be parallel to the proximal end 135 of the two plunger elements 152. Instead, the distal edge 446a and the proximal edge 446b of the upper stabilizer 444 may be parallel to each other and the distal edge 446a and the proximal edge 446b of the lower stabilizer 444 may be parallel to each other but the corresponding edges of the upper and lower stabilizers 444 may not be parallel to one another. Said differently, the upper stabilizer 444 may have a distal tilt while the lower stabilizer 444 may have a proximal tilt.

In some examples, one or both stabilizer elements 444 can have a slit or a channel, thus dividing that arc-shaped stabilizing element into two segments. Nonetheless, even with a slit on one or both stabilizer elements 444, the stabilizing ring 456, which can be a non-continuous ring, similar to a ring with one or more slots formed through the ring, can still provide the restraining surface to prevent the needle guard 132 from early activation during retraction of the needle 108, prior to the needle tip 110 moving proximally of the two distal walls 300, 302 (see FIG. 3). The restraining surface can also be referred to as a restrict point, choke gap, or choke point since it provides a rigid structure that prevents the needle guard 132 from moving proximally thereof unless or until the needle guard 132 first activates and collapses radially to reduce its radial profile to then slip proximally of the choke point. In an example, one or two elbows 304 (FIG. 7) of the needle guard 132 can be restricted by the choke point from moving in the proximal direction until the one or two elbows 304 of the needle guard 132 deflect to reduce radial profile of the needle guard 132. In an example, when the radial profile of the needle guard 132 is reduced, the needle guard 132 can slip through the bore defined by the stabilizing ring 456, from a distal position of the stabilizing ring 456 to a proximal position of the stabilizing ring 456.

The valve opener 134 can be made from a metal material or from a plastic material. When made from a metal material, the valve opener 134 can be formed by bending or deep draw methods and the arc shape cross section of the actuating element 152 can provide added rigidity when pushed by the male Luer tip 500. Each actuating element 152 can comprise at least two lengthwise edges and a rib can be provided along one or both of the lengthwise edges to further add structural rigidity. One or more gaps can be provided between any two actuating elements 152. The gaps can provide clearance or space for fluid flow flowing thereacross, such as during flushing blood or IV infusion. The gap between the actuating elements 152 can define a holding space to accommodate a needle guard 132.

In some embodiments, a majority or most if not all of the needle guard 132 fits within the holding space formed by the body of the valve opener 134, between the two plunger elements 152, in the ready to use position, as shown in FIG. 1. This allows the catheter hub 102 to be more compact, as less longitudinal space is needed within the catheter hub 102 to fit both the actuator 134 and the tip protector 132 serially lengthwise or when the two only partially overlap in the axial direction. As shown in FIG. 1, the proximal wall 280 of the needle guard 132 is generally flush or even with the proximal end surfaces of the two plunger elements 152.

When the needle guard 132 only engages with the distal edge 446a (see FIGS. 4-5) of the relief or through passage 448 (see FIGS. 4-5) in the valve opener 134, then no deformity or change of diameter is required on the inside wall of the catheter hub 102 and the needle guard 132 can be placed further proximally in the female Luer taper section while complying with the international Luer standard for conical fittings and the overall length of the catheter hub 102 can be further reduced.

Referring again to FIGS. 4-5, when initially inserting the male medical implement, such as a male Luer tip 500, into the proximal opening of the catheter hub 102, the male Luer tip 500 initially contacts the two actuating elements 152 on the valve opener 134 to advance a distally directed force on the two actuating elements 152 to open the valve 136. The distally directed force moves the valve opener 134 in the distal direction until the geometries of the male Luer tip 500 and the proximal opening 121 of the catheter hub 102 stop further distal advancement of the male Luer tip 500. In an example, a female Luer taper of the catheter hub 102 and a male Luer taper of the male Luer tip 500 mate and block distal advancement of the male Luer tip 500 further into the opening of the catheter hub 102. A seal is provided by the Luer engagement to prevent fluid from leaking out the proximal opening 121 of the catheter hub 102.

As the valve opener 134 moves distally by the distal advancement of the male Luer tip 500, the nose section 430 of the valve actuator 134 is urged distally and pushes against the proximal end 141 of the valve 136. In particular, the nose section 430 of the valve opener 134 initially pushes against the proximal end 141 of the valve 136 and since the valve 136 is axially fixed within the first recessed section 420 of the catheter hub 102, the one or more flaps 145 on the valve 136 deflect radially and in the distal direction. A reversible or internal force may be created within the valve 136 upon the one or more flaps 145 being pushed. Fluid from the male Luer tip 500 can then flow through the catheter hub 102, through the valve 136, and through the lumen of the catheter tube 104.

Alternatively, a suction can be applied by the male medical instrument, such as a syringe or vacuum blood collection tube, and blood aspirated from the patient. This is often done for testing samples before infusion therapy commences. Also, typically any remaining blood is first flushed from the inside of the catheter hub 102 before infusion therapy commences.

After removal of the male Luer tip 500, the valve opener 134 may disengage from the valve 136. The flaps 145 may then return to their un-deformed position due to the internal force initially created by opening of the valve 136. Hence, the valve 136 may be used or actuated multiple times, the valve opener 134 and valve 136 returning to the pre-activation position shown in FIG. 1 when the male tip is removed.

As shown in FIG. 4, the nose section 430 may be configured so that when pushed into the valve 136 during activation, the actuation or activation end 436 does not extend distally past the flaps 145 of the valve 136. This configuration can ensure that the valve opener 134 is pushed back by the flaps 145 when the male Luer tip 500 is removed. The distal movement of the male Luer tip 500 is a minimum distal movement. In other embodiments, such as that of FIG. 5, there may be a maximum distal movement. The difference between the maximum movement and minimum movement of a standard Luer connector may be approximately 2.5 millimeters.

Methods of making and of using embodiments of the catheter assemblies and components thereof described herein are within the scope of the present invention.

Needle assemblies, catheter assemblies, components of needle and catheter assemblies, and method for using and making catheter assemblies or needle assemblies in accordance with the invention may be covered by the following exemplary aspects:

Exemplary aspect 1: A needle assembly comprising:
a needle hub with a hub body and a needle with a needle tip extending from a distal end of the hub body;
a catheter tube attached to a catheter hub and having the needle extending through the catheter tube in a ready to use position;
a valve positioned in an interior cavity of the catheter hub, the valve comprising a valve body having an outer perimeter, an interior surface defining an interior cavity, a distal opening, a wall structure at a proximal end of the valve body, at least one slit and a plurality of flaps at the wall structure, and at least one interior bump on each of the plurality of flaps, each interior bump extending axially distally from a distally facing wall surface of the wall structure in interior cavity of the valve; and
a valve opener comprising a nose section with a push end sized and shaped to open the at least one slit to open the valve when displaced distally by a male Luer tip.
   Exemplary aspect 2: The needle assembly of aspect 1, further comprising a plurality of exterior bumps extending in a proximal direction from a proximally facing surface of the wall structure.
   Exemplary aspect 3: The needle assembly of any one of aspects 1 or 2, wherein each of the plurality of flaps has at least one exterior bump.
   Exemplary aspect 4: The needle assembly of any one of aspects 1 to 3, wherein the valve comprises a slanted wall surface located in the interior cavity at a location to be contacted by the at least one interior bump when the plurality of flaps is deflected by the valve opener.
   Exemplary aspect 5: The needle assembly of any one of aspects 1 to 4, wherein the interior cavity has a first generally cylindrical wall section located adjacent the slanted wall surface and a second generally cylindrical wall section located adjacent the slanted wall surface and remote from the first generally cylindrical wall section.
   Exemplary aspect 6: The needle assembly of any one of aspects 1 to 5, wherein the first generally cylindrical wall section has a first inside diameter and wherein the second generally cylindrical wall section has a second inside diameter, and wherein the first inside diameter is larger than the second inside diameter.
   Exemplary aspect 7: The needle assembly of any one of aspects 1 to 6, wherein the distal opening opens into the second generally cylindrical wall section when viewing in a proximal direction from the distal opening.
   Exemplary aspect 8: The needle assembly of any one of aspects 1 to 7, wherein the at least one slit comprises three slits that converge at a central location and the plurality of flaps comprise three flaps.
   Exemplary aspect 9: The needle assembly of any one of aspects 1 to 8, wherein the valve opener comprises two actuating elements having the needle guard located therebetween and a stabilizer connecting the two actuating elements.
   Exemplary aspect 10: The needle assembly of any one of aspects 1 to 9, wherein the distal opening is at least partially aligned at a central axis of the valve body.
   Exemplary aspect 11: The needle assembly of any one of aspects 1 to 10, wherein the interior cavity has an inside diameter, and wherein the distal opening has an opening diameter that is smaller than the inside diameter.
   Exemplary aspect 12: The needle assembly of any one of aspects 1 to 11, wherein a distal wall on the valve comprises a distally facing wall surface and a proximally facing wall surface, and wherein the distal opening is formed through the distal wall.
   Exemplary aspect 13: The needle assembly of any one of aspects 1 to 12, can further comprise a needle guard positioned in the interior cavity of the catheter hub and placed over the needle, the needle guard comprising a wall positioned to a side of the needle and movable distal of the needle tip to block the needle tip from inadvertent needlesticks in a protective position.
   Exemplary aspect 14: The needle assembly of any one of aspects 1 to 13, wherein the valve may be practiced with the interior bumps only, without also the exterior bumps. The wall structure without the exterior bumps is generally flat or planar.
   Exemplary aspect 15: The needle assembly of any one of aspects 1 to 14, wherein the valve may further comprise a plurality of exterior bumps extending in a proximal direction from a proximally facing surface of the wall structure.
   Exemplary 16: The needle assembly of any one of aspects 1 to 15, wherein each of the plurality of flaps may have at least one exterior bump located thereon.
   Exemplary aspect 17: The needle assembly of any one of aspects 1 to 16, wherein either the interior bumps, the exterior bumps, or both the interior and exterior bumps on the wall structure at the proximal end of the valve, such as on the flaps of the wall structure, can extend or increase the thickness of the wall structure.
   Exemplary aspect 18: The needle assembly of any one of aspects 1 to 17, wherein the thickness of the wall structure can be measured from the exterior most surface at the proximal side to the exterior most surface at the distal side of the wall structure, where the flaps are provided.
   Exemplary aspect 19: The needle assembly of any one of aspects 1 to 18, wherein when the thickness of the wall structure is extended or increased by the thickness of the interior and/or exterior bumps, the flaps can be deflected by a valve opener with a male medical implement without having to deflect the flaps the same amount compared to when no bumps are incorporated on the wall structure.
   Exemplary aspect 20: The needle assembly of any one of aspects 1 to 19, wherein the valve flaps with interior and/or exterior bumps are deflected by the valve opener, the valve flaps can contact an interior surface of the valve body, such as the interior surface that forms the interior cavity.
   Exemplary aspect 21: The needle assembly of any one of aspects 1 to 20, wherein the valve body comprises a first generally cylindrical wall section with a first inside diameter and a second generally cylindrical wall section with a second inside diameter, and wherein the first inside diameter is larger than the second inside diameter.
   Exemplary aspect 22: The needle assembly of any one of aspects 1 to 21, wherein the valve body comprises a first generally cylindrical wall section with a first inside diameter and a second generally cylindrical wall section with a second inside diameter, and wherein the first inside diameter is smaller than the second inside diameter.
   Exemplary aspect 23: The needle assembly of any one of aspects 1 to 22, wherein the valve body can have a second generally cylindrical wall section located distally of a first generally cylindrical wall section.
   Exemplary aspect 24: The needle assembly of any one of aspects 1 to 23, wherein the valve opener comprises two actuating elements having a needle guard located therebetween and a stabilizer connecting the two actuating elements.
   Exemplary aspect 25: The needle assembly of any one of aspects 1 to 24, wherein the valve opener comprises a nose section with an actuation end and wherein the actuation end contacts the valve in the ready to use position.
   Exemplary aspect 26: The needle assembly of any one of aspects 1 to 25, wherein the valve opener comprises a nose section with an actuation end and wherein the actuation end is spaced from the valve in the ready to use position.
   Exemplary aspect 27: The needle assembly of any one of aspects 1 to 26, wherein the proximal opening of the catheter hub has a Luer opening, optionally with or without external threads.
   Exemplary aspect 28: The needle assembly of any one of aspects 1 to 27, wherein the wall structure of the valve is oriented proximally of the distal opening when the valve is mounted inside the catheter hub.
   Exemplary aspect 29: The needle assembly of any one of aspects 1 to 28, wherein a plurality of slits is located between a plurality of exterior bumps.
   Exemplary aspect 30: The needle assembly of any one of aspects 1 to 29, wherein a wall structure on the valve has a planar surface, and wherein the plurality of exterior bumps extends proximally of the planar surface.
   Exemplary aspect 31: The needle assembly of any one of aspects 1 to 30, wherein the catheter hub has a groove and wherein the valve body is located within the groove.
   Exemplary aspect 32: The needle assembly of any one of aspects 1 to 31, wherein the exterior bumps can each comprise an arcuate shape with rounded edges resembling radially cut segments of a donut or segments of a torus.
   Exemplary aspect 33: The needle assembly of any one of aspects 1 to 32, wherein the exterior bumps can each have a different shape from other exterior bumps, or all exterior bumps can have the same shape.
   Exemplary aspect 34: The needle assembly of any one of aspects 1 to 33, wherein the exterior bump can have a circular shape, a square shape, a rectangular shape, a triangular shape, a segment of a torus, or an irregular shaped cross-section.
   Exemplary aspect 35: The needle assembly of any one of aspects 1 to 34, wherein the exterior bumps can be omitted from the flaps and the valve comprising interior bumps only.
   Exemplary aspect 36: The needle assembly of any one of aspects 1 to 35, wherein the number of exterior bumps is the same or smaller than the number of flaps.
   Exemplary aspect 37: The needle assembly of any one of aspects 1 to 36, wherein the interior bumps can each comprise an arcuate shape with rounded edges resembling radially cut segments of a donut or segments of a torus.
   Exemplary aspect 38: The needle assembly of any one of aspects 1 to 37, wherein the interior bumps can each have a different shape from other interior bumps, or all interior bumps can have the same shape.
   Exemplary aspect 39: The needle assembly of any one of aspects 1 to 38, wherein the interior bump can have a circular shape, a square shape, a rectangular shape, a triangular shape, a segment of a torus, or an irregular shaped cross-section.
   Exemplary aspect 40: The needle assembly of any one of aspects 1 to 39, wherein the interior bumps can be omitted from the flaps and the valve comprising exterior bumps only.
   Exemplary aspect 41: The needle assembly of any one of aspects 1 to 40, wherein the number of exterior bumps is the same or smaller than the number of flaps.
   Exemplary aspect 42: The needle assembly of any one of aspects 1 to 41, wherein each pair of interior bump and exterior bump is aligned at the wall structure.
   Exemplary aspect 43: The needle assembly of any one of aspects 1 to 42, wherein each pair of interior bump and exterior bump is offset and not aligned at the wall structure.
   Exemplary aspect 44: The needle assembly of any one of aspects 1 to 43, wherein the interior bumps make contact with an interior wall that defines part of the interior cavity of the valve when the flaps of the valve are opened by the valve opener.
   Exemplary aspect 45: The needle assembly of any one of aspects 1 to 44, wherein the valve opener is advanced in the distal direction by a male Luer tip.
   Exemplary aspect 46: The needle assembly of any one of aspects 1 to 45, wherein the size of the interior bumps and the geometry of the interior wall can be selected to control the amount of compression of the flaps and the interior bumps against the interior wall of the valve body when the flaps are deflected by the valve opener.
   Exemplary aspect 47: The needle assembly of any one of aspects 1 to 46, wherein a restoring force for the flaps to return to their closed position upon removal of the male Luer tip is generated by compressing the interior bumps against the interior wall.
   Exemplary aspect 48: The needle assembly of any one of aspects 1 to 47, wherein the interior bumps are energizers to aid the flaps to return to their closed position to close the valve and limit or restrict flow across the valve.
   Exemplary aspect 49: The needle assembly of any one of aspects 1 to 48, wherein the energizers prevent or delay plastic deformation and thermal setting.
   Exemplary aspect 50: The needle assembly of any one of aspects 1 to 49, wherein the valve has an interior surface defining an interior cavity and an internal wall that modifies a contour of the interior surface.
   Exemplary aspect 51. The needle assembly of any one of aspects 1 to 50, wherein the internal wall of the valve comprises a curved surface that is concaving in a distal direction.
   Exemplary aspect 52: The needle assembly of any one of aspects 1 to 51, wherein the tapered wall has a minimum diameter along its distal side or end, the minimum diameter defining an opening that is sized and shaped to accommodate the needle.
   Exemplary aspect 53: The needle assembly of any one of aspects 1 to 52, wherein the bore section is coincident with a minimum diameter of the internal wall.
   Exemplary aspect 54: A catheter assembly or needle assembly comprising a valve and a valve opener, and wherein the valve comprises a valve body comprising an elongated section having a distal end and a proximal end, a wall structure at the proximal end, and wherein exterior bumps extend proximally from a proximally facing surface of the wall structure and interior bumps extend distally from a distally facing surface of the wall structure. The wall structure can have a plurality of slits forming a plurality of flaps. Each flap can have at least one exterior bump. Each flap can have at least one interior bump. The flaps can deflect by the valve opener, which can move distally by a male Luer tip inserted into a proximal opening of a catheter hub.
   Exemplary aspect 55: A catheter assembly comprising:
a needle hub with a needle having a needle tip;
a catheter tube attached to a catheter hub having a body comprising an interior surface defining an interior cavity, the needle extending through the catheter tube;
a valve positioned in the interior cavity of the catheter hub, the valve comprising a valve body having an exterior surface and an interior surface defining an interior cavity, a distal opening at a distal end of the valve body and a wall structure at a proximal end of the valve body, the wall structure comprising a plurality of slits defining a plurality of flaps;
a plurality of interior bumps located on the plurality of flaps at a location inside the interior cavity of the valve and a plurality of exterior bumps located on the plurality of flaps at a location outside of the interior cavity of the valve;
a valve opener located inside the interior cavity of the catheter hub and proximal of the valve, the valve opener comprising a nose section having an activation end, at least one plunger element having a proximal end surface for pushing by a male medical implement;
a needle guard having a proximal wall and a blocking wall, wherein the blocking wall is positioned to a side of the needle and movable distal of the needle tip to block the needle tip from inadvertent needlesticks in a protective position; and
   wherein at least one interior bump is aligned with at least one exterior bump to define an aligned bump section, and wherein a thickness measured at the aligned bump section is thicker than a thickness of the wall structure without the aligned bump section.
   Exemplary aspect 56: A method of assembling a needle assembly comprising:
providing a catheter hub with a catheter tube having an open distal end and a proximal end attached to the catheter hub by a bushing, the catheter hub comprising a hub body having an interior surface defining an interior cavity and a proximal opening;
positioning a valve in the interior cavity proximal of the bushing, the valve comprising a valve body having an interior surface defining an interior cavity, a distal opening at a distal end of the valve body and a wall structure at a proximal end of the valve body, at least one slit and a plurality of flaps on the wall structure, and a plurality interior bumps in the interior cavity of the valve with at least one interior bump located on each of the plurality of flaps;
positioning a valve opener inside the interior cavity of the catheter hub and proximal of the valve in a proximal position, the valve opener being distally slidable when pushed by a male medical implement into the valve to deflect the plurality of flaps in a distal direction to compress the plurality of interior bumps against the interior surface of the valve to generate an internal reaction force configured to return the plurality of flaps to a closed position upon proximal movement of the valve opener to the proximal position; and
placing a needle attached to a needle hub through the catheter hub, the valve, and the catheter tube so that a tip of the needle extends out the open distal end of the catheter tube in a ready to use position.
   Exemplary aspect 57: The method of aspect 56, further comprising the step of placing a needle guard inside the interior cavity of the catheter hub for blocking the tip of the needle.
   Exemplary aspect 58: The method of any one of aspects 56 or 57, wherein the at least one slit comprises three slits and the plurality of flaps comprise three flaps.
   Exemplary aspect 59: The method of any one of aspects 56 to 58, wherein the valve opener comprises a nose section with an actuation end and wherein the actuation end contacts the valve in the ready to use position.
   Exemplary aspect 60: The method of any one of aspects 56 to 59, wherein the valve opener comprises a nose section with an actuation end and wherein the actuation end is spaced from the valve in the ready to use position.
   Exemplary aspect 61: The method of any one of aspects 56 to 60, further comprising placing an air vent plug at a proximal opening of the needle hub.
   Exemplary aspect 62: The method of any one of aspects 56 to 61, further comprises placing the needle assembly inside a thermoplastic package and sealing the needle assembly inside the thermoplastic package.

Although limited embodiments of catheter assemblies and their components have been specifically described and illustrated herein, many modifications and variations will be apparent to those skilled in the art. For example, the needle guard may be of one piece or can be integrated from more than one piece, such as from multiple pieces. Furthermore, it is understood and contemplated that features specifically discussed for one catheter assembly or for one component may be adopted for inclusion with another catheter assembly or another component, provided the functions are compatible. Accordingly, it is to be understood that the catheter assemblies and their components constructed according to principles of the disclosed devices, systems, and methods may be embodied other than as specifically described herein. The valve and valve opener described herein can also be used with a needle hub by locating them inside a female Luer taper of the needle hub. The valve and valve opener can also be used in the female connector of an infusion needle or a blood collection device or a central venous catheter or peripherally inserted central catheter (PICC) or an arterial catheter or a dialysis needle. In other words, the valve and valve opener can be used in any medical device intended for infusion or bodily fluid collection with a female Luer housing or hub. The disclosure is also defined in the following claims.

1-20. (canceled)

21. A needle assembly comprising:
a needle hub with a hub body and a needle with a needle tip extending from a distal end of the hub body;
a catheter tube attached to a catheter hub and having the needle extending through the catheter tube in a ready to use position;
a valve positioned in an interior cavity of the catheter hub, the valve comprising a valve body having an outer perimeter, an interior surface defining an interior cavity, a distal opening, a wall structure at a proximal end of the valve body, at least one slit and a plurality of flaps at the wall structure, and at least one interior bump on each of the plurality of flaps, each interior bump extending axially distally from a distally facing wall surface of the wall structure in interior cavity of the valve;
a valve opener comprising a nose section with a push end sized and shaped to open the at least one slit to open the valve when displaced distally by a male Luer tip; and
a needle guard positioned in the interior cavity of the catheter hub and placed over the needle, the needle guard comprising a wall positioned to a side of the needle and movable distal of the needle tip to block the needle tip from inadvertent needlesticks in a protective position.

22. The needle assembly of claim 21, further comprising a plurality of exterior bumps extending in a proximal direction from a proximally facing surface of the wall structure.

23. The needle assembly of claim 22, wherein each of the plurality of flaps has at least one exterior bump.

24. The needle assembly of claim 21, wherein the valve comprises a slanted wall surface located in the interior cavity at a location to be contacted by the at least one interior bump when the plurality of flaps is deflected by the valve opener.

25. The needle assembly of claim 24, wherein the interior cavity has a first generally cylindrical wall section located adjacent the slanted wall surface and a second generally cylindrical wall section located adjacent the slanted wall surface and remote from the first generally cylindrical wall section.

26. The needle assembly of claim 25, wherein the first generally cylindrical wall section has a first inside diameter and wherein the second generally cylindrical wall section has a second inside diameter, and wherein the first inside diameter is larger than the second inside diameter.

27. The needle assembly of claim 25, wherein the distal opening opens into the second generally cylindrical wall section when viewing in a proximal direction from the distal opening.

28. The needle assembly of claim 21, wherein the at least one slit comprises three slits that converge at a central location and the plurality of flaps comprise three flaps.

29. The needle assembly of claim 21, wherein the valve opener comprises two actuating elements having the needle guard located therebetween and a stabilizer connecting the two actuating elements.

30. A catheter assembly comprising:
a needle hub with a needle having a needle tip;
a catheter tube attached to a catheter hub having a body comprising an interior surface defining an interior cavity, the needle extending through the catheter tube;
a valve positioned in the interior cavity of the catheter hub, the valve comprising a valve body having an exterior surface and an interior surface defining an interior cavity, a distal opening at a distal end of the valve body and a wall structure at a proximal end of the valve body, the wall structure comprising a plurality of slits defining a plurality of flaps;
a plurality of interior bumps located on the plurality of flaps at a location inside the interior cavity of the valve and a plurality of exterior bumps located on the plurality of flaps at a location outside of the interior cavity of the valve;
a valve opener located inside the interior cavity of the catheter hub and proximal of the valve, the valve opener comprising a nose section having an activation end, at least one plunger element having a proximal end surface for pushing by a male medical implement;
a needle guard having a proximal wall and a blocking wall, wherein the blocking wall is positioned to a side of the needle and movable distal of the needle tip to block the needle tip from inadvertent needlesticks in a protective position; and
wherein at least one interior bump is aligned with at least one exterior bump to define an aligned bump section, and wherein a thickness measured at the aligned bump section is thicker than a thickness of the wall structure without the aligned bump section.

31. The catheter assembly of claim 30, wherein each of the plurality of flaps has at least one interior bump aligned with at least one exterior bump.

32. The catheter assembly of claim 30, wherein the plurality of slits comprise three slits and the plurality of flaps comprise three flaps.

33. The catheter assembly of claim 31, wherein the valve comprises a slanted wall surface located in the interior cavity at a location to be contacted by at least one of the plurality of interior bumps when the plurality of flaps is deflected by the valve opener for contacting by the interior bumps.

34. The catheter assembly of claim 30, wherein the interior cavity of the valve has a first generally cylindrical wall section located adjacent the slanted wall surface and a second generally cylindrical wall section located adjacent the slanted wall surface and remote from the first generally cylindrical wall section.

35. The catheter assembly of claim 34, wherein the distal opening opens into the second generally cylindrical wall section when viewing in a proximal direction from the distal opening.

36. The catheter assembly claim 30, wherein the valve opener further comprises a stabilizing ring defining a continuous perimeter section.

37. A method of assembling a needle assembly comprising:
providing a catheter hub with a catheter tube having an open distal end and a proximal end attached to the catheter hub by a bushing, the catheter hub comprising a hub body having an interior surface defining an interior cavity and a proximal opening;
positioning a valve in the interior cavity proximal of the bushing, the valve comprising a valve body having an interior surface defining an interior cavity, a distal opening at a distal end of the valve body and a wall structure at a proximal end of the valve body, at least one slit and a plurality of flaps on the wall structure, and a plurality interior bumps in the interior cavity of the valve with at least one interior bump located on each of the plurality of flaps;
positioning a valve opener inside the interior cavity of the catheter hub and proximal of the valve in a proximal position, the valve opener being distally slidable when pushed by a male medical implement into the valve to deflect the plurality of flaps in a distal direction to compress the plurality of interior bumps against the interior surface of the valve to generate an internal reaction force configured to return the plurality of flaps to a closed position upon proximal movement of the valve opener to the proximal position;
placing a needle attached to a needle hub through the catheter hub, the valve, and the catheter tube so that a tip of the needle extends out the open distal end of the catheter tube in a ready to use position; and
placing a needle guard inside the interior cavity of the catheter hub for blocking the tip of the needle.

38. The method of claim 37, wherein the at least one slit comprises three slits and the plurality of flaps comprise three flaps.

39. The method of claim 37, wherein the valve opener comprises a nose section with an actuation end and wherein the actuation end contacts the valve in the ready to use position.

40. The method of claim 37, wherein the valve opener comprises a nose section with an actuation end and wherein the actuation end is spaced from the valve in the ready to use position.
